(19) United States
(12) Patent Application Publication
(10) Pub. No.: US 2026/0216064 A1
(43) Pub. Date: Jul. 30, 2026

(54) Novel Composition

(71) Applicant: RB Health (US) LLC, Parsippany, NJ (US)

(72) Inventors: Laurence Arthur Hunt, Hull (GB); Sophie Levesley, Hull (GB); Philip Smith, Hull (GB)

(73) Assignee: RB Health (US) LLC, Parsippany, NJ (US)

(21) Appl. No.: 19/141,558

(22) PCT Filed: Dec. 19, 2023
(86) PCT No.: PCT/GB2023/053305

§ 371 Date: Jun. 20, 2025

(30) Foreign Application Priority Data
Dec. 21, 2022 (GB) 2219376.7

(51) Int. Cl. A61K 9/00; A61K 31/167; A61K 31/485; A61K 31/519; A61K 47/32

(52) U.S. Cl. CPC A61K 9/0056; A61K 31/167; A61K 31/485; A61K 31/519; A61K 47/32

(57) ABSTRACT

A composition including a combination of a first polymer, a second polymer and an active pharmaceutical ingredient and pharmaceutically acceptable salts or esters thereof wherein the active pharmaceutical ingredient can be selected from expectorants, antitussives, mucolytics, erectile dysfunction medicaments and analgesics other than NSAIDs and wherein the active pharmaceutical ingredient and the combination of the first polymer and the second polymer are in the form of a solidified melt extrudate. The combination of the first and second polymer act as a taste-masking agent for the active pharmaceutical ingredient.

The present invention is directed to the use of a combination of polymers to improve the palatability of active pharmaceutical ingredients. In particular, the present invention is directed to the use of a combination of polymers to improve the palatability of water-soluble active pharmaceutical ingredients selected from antitussives, expectorants, mucolytics, erectile dysfunction medicaments and analgesics other than NSAIDs, expectorants.

There are several routes for the administration of active pharmaceutical ingredients (APIs) to a patient, e.g. orally, parenterally, nasally and transdermally. The oral route is an easy and convenient route and as such remains the most attractive for delivery of APIs. The common oral dosage forms are solutions, suspensions, tablets, caplets, liquid filled gelatin capsules, lozenges and troches.

Many APIs have an unpleasant or bitter taste which can impact on their acceptability in some oral dosage forms. It is well known that these unpleasant or bitter taste characteristics limit their consumer acceptability in direct to mouth formats, for example orally dissolving tablets, lyophilisates, granules and liquids.

Conventional techniques for masking the unpleasant or bitter taste of APIs include the addition of flavours and/or sweeteners to the composition and coating the API with substances which prevent it from contacting the taste buds during oral administration.

In addition, a large excess of taste-masking agent is required to prevent or in some way mitigate the bitter taste of any API that is retained in the oral cavity after the composition has passed from there into the gastro-intestinal tract.

Fast acting forms of APIs with high solubility are known to be particularly challenging to taste mask, and conventional effective methods for masking the unpleasant taste characteristics (for example coatings) often directly impact the release of the API itself, thus negatively impacting the favourable pharmacokinetic characteristics and rendering a fast acting, highly soluble API slower releasing and reducing its performance potential.

Recent advances in technology have presented viable dosage alternatives to taste-mask bitter drugs. Several approaches have been reported which involve complexation, freeze-drying, microencapsulation, fluidized-bed coating and supercritical fluids for taste-masking purposes.

A particular problem exists for compositions that are intended to dissolve or disperse in the oral cavity, such as orodispersible tablets or films.

Many APIs do exist in these formats but they often contain taste masking technology which is either ineffective at taste masking the API itself, or they impact the performance of the API itself.

It would, therefore, be desirable to develop a composition which delivers taste masked, fast acting forms of APIs, without impacting the potential for in-vivo release and absorption.

According to a first aspect of the present invention there is provided the use of a combination of a first polymer and a second polymer as a taste-masking agent for an active pharmaceutical ingredient and pharmaceutically acceptable salts or esters thereof wherein the active pharmaceutical ingredient can be selected from expectorants, antitussives, mucolytics, erectile dysfunction medicaments and analgesics other than NSAIDs, wherein the active pharmaceutical ingredient and the combination of the first polymer and the second polymer are in the form of a solidified melt extrudate.

The analgesics other than NSAIDs can be selected from paracetamol, codeine or dihydrocodeine.

The antitussives can be selected from dextromethorphan and dextromethorphan hydrobromide.

The mucolytics can be selected to be ambroxol hydrochloride.

The expectorants can be selected to be guaifenesin.

The erectile dysfunction medicaments can be selected from sildenafil, vardenafil and tadalafil.

The extrudate can comprise up to 60% by weight of the active pharmaceutical ingredient. The extrudate can comprise up to 55% by weight of the active pharmaceutical ingredient. The extrudate can comprise up to 50% by weight of the active pharmaceutical ingredient. The extrudate can comprise up to 45% by weight of the active pharmaceutical ingredient. The extrudate can comprise up to 40% by weight of the active pharmaceutical ingredient.

The extrudate can comprise at least 10% by weight of the active pharmaceutical ingredient. The extrudate can comprise at least 15% by weight of the active pharmaceutical ingredient. The extrudate can comprise at least 20% by weight of the active pharmaceutical ingredient. The extrudate can comprise at least 25% by weight of the active pharmaceutical ingredient. The extrudate can comprise at least 30% by weight of the active pharmaceutical ingredient.

The extrudate can comprise from 10% by weight to 60% by weight of the active pharmaceutical ingredient. The extrudate can comprise from 15% by weight to 55% by weight of the active pharmaceutical ingredient. The extrudate can comprise from 20% by weight to 50% by weight of the active pharmaceutical ingredient. The extrudate can comprise from 25% by weight to 45% by weight of the active pharmaceutical ingredient. The extrudate can comprise from 30% by weight to 40% by weight of the active pharmaceutical ingredient.

Typically, the combination of polymers is miscible with the active pharmaceutical ingredient.

The first polymer of the combination of at least two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 30° C. and 60° C. The polymer can have a glass transition temperature of between 40° C. and 50° C.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

The first polymer of the combination of two polymers can be present at a level of 10-60% by weight of the extrudate.

The first polymer of the combination of two polymers can be present at a level of 10-25% by weight of the extrudate.

Alternatively, the first polymer of the combination of two polymers can be present at a level of 35-50% by weight of the extrudate.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of less than 140° C. The polymer can have a glass transition temperature of between 80° C. and 140° C. The polymer can have a glass transition temperature of between 90° C. and 110° C.

Alternatively, the second polymer of the combination of two polymers can be selected from polymers having a glass transition temperature of at least 55° C. The polymer can have a glass transition temperature of at least 60° C. The polymer can have a glass transition temperature of at least 65° C. The polymer can have a glass transition temperature of less than 70° C. The polymer can have a glass transition temperature of less than 75° C. The polymer can have a glass transition temperature of less than 80° C. The polymer can have a glass transition temperature of between 55° C. and 80° C. The polymer can have a glass transition temperature of between 60° C. and 75° C. The polymer can have a glass transition temperature of between 62° C. and 70° C.

The solidified melt extrudate can comprise an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C. More preferably, the solidified melt extrudate comprises an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can consist essentially of an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C.

The solidified melt extrudate can consist essentially of an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can comprise an analgesic other than an NSAID, a combination of two polymers wherein the first polymer can have a glass transition temperature of between 30° C. and 60° C. and the second polymer can have a glass transition temperature of between 55° C. and 80° C. More preferably, the solidified melt extrudate comprises an analgesic other than an NSAID, a combination of two polymers wherein the first polymer can have a glass transition temperature of between 40° C. and 50° C. and the second polymer can have a glass transition temperature of between 65° C. and 70° C.

The solidified melt extrudate can consist essentially of an analgesic other than an NSAID, a combination of two polymers wherein the first polymer can have a glass transition temperature of between 30° C. and 60° C. and the second polymer can have a glass transition temperature of between 55° C. and 80° C.

The solidified melt extrudate can consist essentially of an analgesic other than an NSAID, a combination of two polymers wherein the first polymer can have a glass transition temperature of between 40° C. and 50° C. and the second polymer can have a glass transition temperature of between 65° C. and 70° C.

The second polymer of the combination of two polymers can be selected from hydroxypropylmethylcelluloses, polyoxyethylene-polypropylene glycols (also known as poloxamers), polyvinylpyrrolidone, polyvinylpyrrolidone-vinyl acetate co-polymer and polyvinyl caprolactam-polyvinyl acetate-polyethylene glycol graft copolymer and polyvinyl alcohol. The second polymer can be hydroxypropylmethyl cellulose, hydroxypropylmethyl cellulose acetate succinate, poloxamer 188, poloxamer 407, polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons. Preferred polymers are polyvinylpyrrolidone K12, polyvinylpyrrolidone-vinyl acetate co-polymer (PVPVA64) or a polyvinyl alcohol having a molecular weight of 30,000-35,000 Daltons.

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons.

The second polymer is selected such that the extrudate has a tensile strength of 5-50 N/mm².

The second polymer of the combination of two polymers can be present at a level of 10-60% by weight of the extrudate.

The second polymer of the combination of two polymers can be present at a level of 10-25% by weight of the extrudate. The second polymer of the combination of two polymers can be present at a level of 20-25% by weight of the extrudate.

Alternatively, the second polymer of the combination of two polymers can be present at a level of 30-60% by weight of the extrudate. The second polymer of the combination of two polymers can be present at a level of 50-60% by weight of the extrudate.

The combination of the first and second polymers can be dimethylaminoethyl methacrylate co-polymer and polyvinylpyrrolidone-vinyl acetate co-polymer (PVPVA64). The combination of first and second polymers is dimethylaminoethyl methacrylate co-polymer and polyvinylpyrrolidone K12. The combination of the first and second polymers can be dimethylaminoethyl methacrylate co-polymer and polyvinyl alcohol.

The solidified melt extrudate can comprise 10%-60% by weight of an active pharmaceutical ingredient selected from expectorants, antitussives, mucolytics, erectile dysfunction medicaments and analgesics other than NSAIDs and a combination of 10%-60% by weight of the first polymer and 10%-60% by weight of the second polymer.

The ratio of the first polymer and the second polymer of the combination of two polymers can be selected to be from 6:1 to 1:6. The ratio of the first polymer and the second polymer of the combination of two polymers can be selected to be from 5:1 to 1:5. The ratio of the first polymer and the second polymer of the combination of two polymers can be selected to be from 1:1 to 1:3. Alternatively, the ratio of the first polymer to the second polymer can be selected to be 1:4 to 1:5.

The ratio of the of the active pharmaceutical ingredient, the first polymer and the second polymer can be from 1:0.2:0.2-1:2:2.

The ratio of the of the active pharmaceutical ingredient, the first polymer and the second polymer can be from 1:0.2:0.8-1:0.8:0.2. The ratio of the of the active pharmaceutical ingredient, the first polymer and the second polymer can be about 1:0.4:0.6.

Alternatively, the ratio of the of the active pharmaceutical ingredient, the first polymer and the second polymer can be from 1:0.5:1.5-1:0.8:1.8.

Alternatively, the ratio of the of the active pharmaceutical ingredient, the first polymer and the second polymer can be from 1:1.5:1.5-1:1.5:1.5. The ratio of the of the active pharmaceutical ingredient, the first polymer and the second polymer can be about 1:1.16:1.16.

The ratio of the of the active pharmaceutical ingredient, the first polymer and the second polymer can be from 1:0.45:0.35-1:0.65:0.55. The active pharmaceutical ingredient can be selected to be paracetamol, dextromethorphan or sildenafil. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:1.2:0.2-1:1.3:0.3. The active pharmaceutical ingredient can be selected to be paracetamol or dextromethorphan. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:0.2:1.2-1:0.7:1.7. The active pharmaceutical ingredient can be selected to be guaifenesin or paracetamol. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol and a combination of 10%-50% by weight of the first polymer and 10%-50% by weight of the second polymer. The solidified melt extrudate can comprise 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of guaifenesin and a combination of 15%-25% by weight of the first polymer and 45%-55% by weight of the second polymer. The solidified melt extrudate can comprise 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can comprise 40%-50% by weight of dextromethorphan and a combination of 20%-50% by weight of the first polymer and 10%-30% by weight of the second polymer. The solidified melt extrudate can comprise 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of sildenafil and a combination of 20%-40% by weight of the first polymer and 30%-40% by weight of the second polymer. The solidified melt extrudate can comprise 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can consist essentially of 40%-50% by weight of paracetamol and a combination of 10%-50% by weight of the first polymer and 10%-50% by weight of the second polymer. The solidified melt extrudate can consist essentially 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of guaifenesin and a combination of 15%-25% by weight of the first polymer and 45%-55% by weight of the second polymer. The solidified melt extrudate can consist essentially 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can consist essentially of 40%-50% by weight of dextromethorphan and a combination of 20%-50% by weight of the first polymer and 10%-30% by weight of the second polymer. The solidified melt extrudate can consist essentially 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of sildenafil and a combination of 20%-40% by weight of the first polymer and 30%-40% by weight of the second polymer. The solidified melt extrudate can consist essentially 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of the first polymer and 10%-50% by weight of the second polymer.

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol as the sole active pharmaceutical ingredient and a combination of 10%-50% by weight of the first polymer and 10%-50% by weight of the second polymer. The solidified melt extrudate can comprise 40%-50% by weight of paracetamol as the sole active pharmaceutical ingredient.

The composition can include one or more excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid and acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone, microcrystalline cellulose, sodium starch glycolate and croscarmellose sodium.

The surfactants can be selected from polysorbates, sodium lauryl sulphate, poloxamers or lecithin. A preferred surfactant is polysorbate 80 (Tween 80). Polysorbate 80 can be present as a level of 1-5 w/w %.

One or more processing aids such as talc, magnesium silicate and glyceryl monostearate can be added prior to melt extrusion or post-melt extrusion.

One or more additional processing aids can be added post-melt extrusion selected from microcrystalline cellulose, crospovidone, carrageenan, chitosan, pectinic acid, glycerides, beta-cyclodextrin and cellulose derivatives, sorbitol and mannitol and isomers thereof.

The combination of polymers is soluble at a pH of about 1 to about 5. Preferably, the combination of polymers is soluble at a pH of about 2 to about 4.

Preferably the difference in Hansen solubility parameter between the active pharmaceutical ingredient and one or both of the first polymer and the second polymer is less than 10 MPa^{1/2}. More preferably the difference in Hansen solubility parameter between the active pharmaceutical ingredient and one or both of the first polymer and the second polymer is less than 7 MPa^{1/2}.

The extrudate is typically prepared using a hot melt extrusion process.

When the active pharmaceutical ingredient is selected from expectorants the melt extrusion process can be conducted at a temperature of between 70° C. and 85° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 70° C. to 85° C. and at an extruder screw speed from 90 rpm to 110 rpm.

When the active pharmaceutical ingredient is selected from antitussives the melt extrusion process can be conducted at a temperature of between 100° C. and 120° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 100° C. to 120° C. and at an extruder screw speed from 90 rpm to 110 rpm.

When the active pharmaceutical ingredient is selected from erectile dysfunction medicaments, the melt extrusion process can be conducted at a temperature of between 110° C. and 140° C. The screw speed of the extruder can be selected from 190 revolutions per minute (rpm) to 210 revolutions per minute (rpm). The screw speed of the extruder can be selected to be about 200 rpm. The melt extrusion process can be carried out at a preferred temperature from 110° C. to 140° C. and at an extruder screw speed from 190 rpm to 210 rpm.

When the active pharmaceutical ingredient is selected from analgesics other than NSAIDs, the melt extrusion process can be conducted at a temperature of between 110° C. and 150° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 110° C. to 150° C. and at an extruder screw speed from 90 rpm to 110 rpm.

The extrusion processing conditions can be selected such that the torque is less than or equal to 15 Nm. The extrusion conditions can be selected such that the torque is more than or equal to 3 Nm.

Preferably the extrusion conditions are selected such that the torque is less than or equal to 15 Nm and equal to or more than 3 Nm.

The solidified melt extrudate comprises an active pharmaceutical ingredient selected from expectorants and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from expectorants and wherein the extrudate can be prepared using a hot melt extrusion process at a temperature between 70° C. and 85° C. and wherein the extruder screw speed can be about 100 rpm.

The solidified melt extrudate comprises an active pharmaceutical ingredient selected from antitussives and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from antitussives and wherein the extrudate can be prepared using a hot melt extrusion process at a temperature between 100° C. and 120° C. and wherein the extruder screw speed can be about 100 rpm.

The solidified melt extrudate comprises an active pharmaceutical ingredient selected from erectile dysfunction medicaments and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from erectile dysfunction medicaments and wherein the extrudate can be prepared using a hot melt extrusion process at a temperature from 110° C. to 140° C. and at an extruder screw speed from 190 rpm to 210 rpm.

The solidified melt extrudate comprises an analgesic other than an NSAID and a combination of dimethylaminoethyl methacrylate co-polymer and polyvinyl alcohol wherein the extrudate can comprise from 30%-50% by weight of the analgesic other than an NSAID wherein the extrudate can be prepared using a hot melt extrusion process at a temperature between 110° C. and 150° C. and wherein the extruder screw speed can be about 100 rpm.

The extrudate can be comminuted into granules that are suitable for incorporation into an oral dosage form such as oral liquid suspensions, tablets, orodispersible tablets, direct to mouth granules, capsules, lyophilates.

According to a second aspect of the present invention there is provided a taste-masked composition in the form of a solidified melt extrudate comprising an active pharmaceutical ingredient and pharmaceutically acceptable salts or esters thereof and a combination of a first polymer and a second polymer wherein the composition has a solubility of less than 5% after 5 mins in an aqueous medium at a pH of between 6 and 7.5 and a solubility of more than 5% after 5 mins in an aqueous medium at a pH of between 1 and 5 and wherein the active pharmaceutical agent can be selected from expectorants, antitussives, mucolytics, erectile dysfunction medicaments and analgesics other than NSAIDs.

When the active is selected to be an expectorant, the composition can have a solubility of less than 5% after 10 mins in an aqueous medium at a pH of between 6 and 7.5 and a solubility of more than 15% after 10 mins in an aqueous medium at a pH of between 1 and 5. The composition can have a solubility of less than 10% after 15 mins in an aqueous medium at a pH of between 6 and 7.5 and a solubility of more than 20% after 15 mins in an aqueous medium at a pH of between 1 and 5.

When the active is selected to be an analgesic other than an NSAID, the composition can have a solubility of less than 10% after 10 mins in an aqueous medium at a pH of between 6 and 7.5 and a solubility of more than 20% after 10 mins in an aqueous medium at a pH of between 1 and 5.

The analgesics other than NSAIDs can be selected from paracetamol, codeine or dihydrocodeine.

The antitussives can be selected from dextromethorphan and dextromethorphan hydrobromide

The mucolytics can be selected to be ambroxol hydrochloride.

The expectorants can be selected to be guaifenesin.

The erectile dysfunction medicaments can be selected from sildenafil, vardenafil and tadalafil.

The extrudate can comprise up to 60% by weight of the active pharmaceutical ingredient. The extrudate can comprise up to 55% by weight of the active pharmaceutical ingredient. The extrudate can comprise up to 50% by weight of the active pharmaceutical ingredient. The extrudate can comprise up to 45% by weight of the active pharmaceutical ingredient. The extrudate can comprise up to 40% by weight of the active pharmaceutical ingredient.

The extrudate can comprise at least 10% by weight of the active pharmaceutical ingredient. The extrudate can comprise at least 15% by weight of the active pharmaceutical ingredient. The extrudate can comprise at least 20% by weight of the active pharmaceutical ingredient.

The extrudate can comprise at least 25% by weight of the active pharmaceutical ingredient. The extrudate can comprise at least 30% by weight of the active pharmaceutical ingredient.

The extrudate can comprise from 10% by weight to 60% by weight of the active pharmaceutical ingredient. The extrudate can comprise from 15% by weight to 55% by weight of the active pharmaceutical ingredient. The extrudate can comprise from 20% by weight to 50% by weight of the active pharmaceutical ingredient. The extrudate can comprise from 25% by weight to 45% by weight of the active pharmaceutical ingredient. The extrudate can comprise from 30% by weight to 40% by weight of the active pharmaceutical ingredient.

Typically, the combination of polymers is miscible with the active pharmaceutical ingredient.

The first polymer of the combination of two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 30° C. and 60° C. The polymer can have a glass transition temperature of between 40° C. and 50° C.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

The first polymer of the combination of two polymers can be present at a level 10-60% by weight of the extrudate.

The first polymer of the combination of two polymers can be present at a level 10-25% by weight of the extrudate.

Alternatively, the first polymer of the combination of two polymers can be present at a level 35-50% by weight of the extrudate.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of less than 140° C. The polymer can have a glass transition temperature of between 80° C. and 140° C. The polymer can have a glass transition temperature of between 90° C. and 110° C.

Alternatively, the second polymer of the combination of two polymers can be selected from polymers having a glass transition temperature of at least 55° C. The polymer can have a glass transition temperature of at least 60° C. The polymer can have a glass transition temperature of at least 65° C. The polymer can have a glass transition temperature of less than 70° C. The polymer can have a glass transition temperature of less than 75° C. The polymer can have a glass transition temperature of less than 80° C. The polymer can have a glass transition temperature of between 55° C. and 80° C. The polymer can have a glass transition temperature of between 60° C. and 75° C. The polymer can have a glass transition temperature of between 62° C. and 70° C.

The solidified melt extrudate can comprise an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C. More preferably, the solidified melt extrudate comprises an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can consist essentially of an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C.

The solidified melt extrudate can consist essentially of an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can comprise an analgesic other than an NSAID, a combination of two polymers wherein the first polymer can have a glass transition temperature of between 30° C. and 60° C. and the second polymer can have a glass transition temperature of between 55° C. and 80° C. More preferably, the solidified melt extrudate comprises an analgesic other than an NSAID, a combination of two polymers wherein the first polymer can have a glass transition temperature of between 40° C. and 50° C. and the second polymer can have a glass transition temperature of between 65° C. and 70° C.

The solidified melt extrudate can consist essentially of an analgesic other than an NSAID, a combination of two polymers wherein the first polymer can have a glass transition temperature of between 30° C. and 60° C. and the second polymer can have a glass transition temperature of between 55° C. and 80° C.

The solidified melt extrudate can consist essentially of an analgesic other than an NSAID, a combination of two polymers wherein the first polymer can have a glass transition temperature of between 40° C. and 50° C. and the second polymer can have a glass transition temperature of between 65° C. and 70° C.

The second polymer of the combination of two polymers can be selected from hydroxypropylmethylcelluloses, polyoxyethylene-polypropylene glycols (also known as poloxamers), polyvinylpyrrolidone, polyvinylpyrrolidone-vinyl acetate co-polymer and polyvinyl caprolactam-polyvinyl acetate-polyethylene glycol graft copolymer and polyvinyl alcohol. The second polymer can be hydroxypropylmethyl cellulose, hydroxypropylmethyl cellulose acetate succinate, poloxamer 188, poloxamer 407, polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons. Preferred polymers are polyvinylpyrrolidone K12, polyvinylpyrrolidone-vinyl acetate co-polymer (PVPVA64) or a polyvinyl alcohol having a molecular weight of 30,000-35,000 Daltons.

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons.

The second polymer is selected such that the extrudate has a tensile strength of 5-50 N/mm².

The second polymer of the combination of two polymers can be present at a level of 10-60% by weight of the extrudate.

The second polymer of the combination of two polymers can be present at a level of 10-25% by weight of the extrudate. The second polymer of the combination of two polymers can be present at a level of 20-25% by weight of the extrudate.

Alternatively, the second polymer of the combination of two polymers can be present at a level of 30-60% by weight of the extrudate. The second polymer of the combination of two polymers can be present at a level of 50-60% by weight of the extrudate.

The combination of the first and second polymers can be dimethylaminoethyl methacrylate co-polymer and polyvinylpyrrolidone-vinyl acetate co-polymer (PVPVA64). The combination of first and second polymers is dimethylaminoethyl methacrylate co-polymer and polyvinylpyrrolidone K12. The combination of the first and second polymers can be dimethylaminoethyl methacrylate co-polymer and polyvinyl alcohol.

The solidified melt extrudate can comprise 10%-60% by weight of an active pharmaceutical ingredient selected from expectorants, antitussives, mucolytics, erectile dysfunction medicaments and analgesics other than NSAIDs and a combination of 10%-60% by weight of the first polymer and 10%-60% by weight of the second polymer.

The ratio of the first polymer and the second polymer of the combination of two polymers can be selected to be from 6:1 to 1:6. The ratio of the first polymer and the second polymer of the combination of two polymers can be selected to be from 5:1 to 1:5. The ratio of the first polymer and the second polymer of the combination of two polymers can be selected to be from 1:1 to 1:3. Alternatively, the ratio of the first polymer to the second polymer can be selected to be 1:4 to 1:5.

The ratio of the of the active pharmaceutical ingredient, the first polymer and the second polymer can be from 1:0.2:0.2-1:2:2.

The ratio of the of the active pharmaceutical ingredient, the first polymer and the second polymer can be from 1:0.2:0.8-1:0.8:0.2. The ratio of the active pharmaceutical ingredient, the first polymer and the second polymer can be about 1:0.4:0.6.

Alternatively, the ratio of the of the active pharmaceutical ingredient, the first polymer and the second polymer can be from 1:0.5:1.5-1:0.8:1.8.

Alternatively, the ratio of the of the active pharmaceutical ingredient, the first polymer and the second polymer can be from 1:1.5:1.5-1:1.5:1.5. The ratio of the of the active pharmaceutical ingredient, the first polymer and the second polymer can be about 1:1.16:1.16.

The ratio of the of the active pharmaceutical ingredient, the first polymer and the second polymer can be from 1:0.45:0.35-1:0.65:0.55. The active pharmaceutical ingredient can be selected to be paracetamol, dextromethorphan or sildenafil. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:1.2:0.2-1:1.3:0.3. The active pharmaceutical ingredient can be selected to be paracetamol or dextromethorphan. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:0.2:1.2-1:0.7:1.7. The active pharmaceutical ingredient can be selected to be guaifenesin or paracetamol. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:1.1:1.1-1:1.25:1.25. The active pharmaceutical ingredient can be sildenafil. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol and a combination of 10%-50% by weight of the first polymer and 10%-50% by weight of the second polymer. The solidified melt extrudate can comprise 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of guaifenesin and a combination of 15%-25% by weight of the first polymer and 45%-55% by weight of the second polymer. The solidified melt extrudate can comprise 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can comprise 40%-50% by weight of dextromethorphan and a combination of 20%-50% by weight of the first polymer and 10%-30% by weight of the second polymer. The solidified melt extrudate can comprise 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of sildenafil and a combination of 20%-40% by weight of the first polymer and 30%-40% by weight of the second polymer. The solidified melt extrudate can comprise 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can consist essentially of 40%-50% by weight of paracetamol and a combination of 10%-50% by weight of the first polymer and 10%-50% by weight of the second polymer. The solidified melt extrudate can consist essentially 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of guaifenesin and a combination of 15%-25% by weight of the first polymer and 45%-55% by weight of the second polymer. The solidified melt extrudate can consist essentially 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can consist essentially of 40%-50% by weight of dextromethorphan and a combination of 20%-50% by weight of the first polymer and 10%-30% by weight of the second polymer. The solidified melt extrudate can consist essentially 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of sildenafil and a combination of 20%-40% by weight of the first polymer and 30%-40% by weight of the second polymer. The solidified melt extrudate can consist essentially 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of the first polymer and 10%-50% by weight of the second polymer.

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol as the sole active pharmaceutical ingredient and a combination of 10%-50% by weight of the first polymer and 10%-50% by weight of the second polymer. The solidified melt extrudate can comprise 40%-50% by weight of paracetamol as the sole active pharmaceutical ingredient.

The composition can include one or more processing aids or excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid and acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone, microcrystalline cellulose, sodium starch glycolate and croscarmellose sodium.

The surfactants can be selected from polysorbates, sodium lauryl sulphate, poloxamers or lecithin. A preferred surfactant is polysorbate 80 (Tween 80). Polysorbate 80 can be present as a level of 1-3 w/w %.

One or more processing aids or excipients such as talc, magnesium silicate and glyceryl monostearate can be added prior to melt extrusion.

One or more additional processing aids or excipients can be added post-melt extrusion selected from microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 2 to about 4.

Preferably the difference in Hansen solubility parameter between the active pharmaceutical ingredient and one or both of the first polymer and the second polymer is less than 10 MPa^{1/2}. More preferably the difference in Hansen solubility parameter between the active pharmaceutical ingredient and one or both of the combination of the first polymer and the second polymer is less than 7 MPa^{1/2}.

The extrudate is typically prepared using a hot melt extrusion process.

When the active pharmaceutical ingredient is selected from expectorants the melt extrusion process can be conducted at a temperature of between 70° C. and 85° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 70° C. to 85° C. and at an extruder screw speed from 90 rpm to 110 rpm.

When the active pharmaceutical ingredient is selected from antitussives the melt extrusion process can be conducted at a temperature of between 100° C. and 120° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 100° C. to 120° C. and at an extruder screw speed from 90 rpm to 110 rpm.

When the active pharmaceutical ingredient is selected from erectile dysfunction medicaments, the melt extrusion process can be conducted at a temperature of between 110° C. and 140° C. The screw speed of the extruder can be selected from 190 revolutions per minute (rpm) to 210 revolutions per minute (rpm). The screw speed of the extruder can be selected to be about 200 rpm. The melt extrusion process can be carried out at a preferred temperature from 110° C. to 140° C. and at an extruder screw speed from 190 rpm to 210 rpm.

When the active pharmaceutical ingredient is selected from analgesics other than NSAIDs, the melt extrusion process can be conducted at a temperature of between 110° C. and 150° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 110° C. to 150° C. and at an extruder screw speed from 90 rpm to 110 rpm.

The extrusion processing conditions can be selected such that the torque is less than or equal to 15 Nm. The extrusion conditions can be selected such that the torque is more than or equal to 3 Nm.

Preferably the extrusion conditions are selected such that the torque is less than or equal to 15 Nm and equal to or more than 3 Nm.

The solidified melt extrudate comprises an active pharmaceutical ingredient selected from expectorants and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from expectorants and wherein the extrudate can be prepared using a hot melt extrusion process at a temperature between 70° C. and 85° C. and wherein the extruder screw speed can be about 100 rpm.

The solidified melt extrudate comprises an active pharmaceutical ingredient selected from antitussives and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from antitussives and wherein the extrudate can be prepared using a hot melt extrusion process at a temperature between 100° C. and 120° C. and wherein the extruder screw speed can be about 100 rpm.

The solidified melt extrudate comprises an active pharmaceutical ingredient selected from erectile dysfunction medicaments and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from erectile dysfunction medicaments and wherein the extrudate can be prepared using a hot melt extrusion process at a temperature from 110° C. to 140° C. and at an extruder screw speed from 190 rpm to 210 rpm.

The solidified melt extrudate comprises an analgesic other than an NSAID and a combination of dimethylaminoethyl methacrylate co-polymer and polyvinyl alcohol wherein the extrudate can comprise from 30%-50% by weight of the analgesic other than an NSAID wherein the extrudate can be prepared using a hot melt extrusion process at a temperature between 110° C. and 150° C. and wherein the extruder screw speed can be about 100 rpm.

The extrudate can be comminuted into granules that are suitable for incorporation into an oral dosage form such as oral liquid suspensions, tablets, orodispersible tablets, direct to mouth granules, capsules and lyophilates.

According to a third aspect of the present invention there is provided a method of taste-masking an active pharmaceutical ingredient by using a combination of a first polymer and a second polymer as described in any of the previous aspects of the present invention wherein the active pharmaceutical agent can be selected from expectorants, antitussives, mucolytics, erectile dysfunction medicaments and analgesics other than NSAIDs, wherein the active pharmaceutical ingredient and the combination of the first polymer and the second polymer are in the form of a solidified melt extrudate.

According to a fourth aspect of the present invention there is provided a composition comprising a combination of a first polymer and a second polymer and an active pharmaceutical ingredient and pharmaceutically acceptable salts or esters thereof wherein the active pharmaceutical ingredient can be selected from expectorants, antitussives, mucolytics, erectile dysfunction medicaments and analgesics other than NSAIDs, wherein the active pharmaceutical ingredient and the combination of the first polymer and the second polymer are in the form of a solidified melt extrudate.

The analgesics other than NSAIDs can be selected from paracetamol, codeine or dihydrocodeine.

The antitussives can be selected from dextromethorphan and dextromethorphan hydrobromide.

The mucolytics can be selected to be ambroxol hydrochloride.

The expectorants can be selected to be guaifenesin.

The erectile dysfunction medicaments can be selected from sildenafil, vardenafil and tadalafil.

The extrudate can comprise up to 60% by weight of the active pharmaceutical ingredient. The extrudate can comprise up to 55% by weight of the active pharmaceutical ingredient. The extrudate can comprise up to 50% by weight of the active pharmaceutical ingredient. The extrudate can comprise up to 45% by weight of the active pharmaceutical ingredient. The extrudate can comprise up to 40% by weight of the active pharmaceutical ingredient.

The extrudate can comprise at least 10% by weight of the active pharmaceutical ingredient. The extrudate can comprise at least 15% by weight of the active pharmaceutical ingredient. The extrudate can comprise at least 20% by weight of the active pharmaceutical ingredient. The extrudate can comprise at least 25% by weight of the active pharmaceutical ingredient. The extrudate can comprise at least 30% by weight of the active pharmaceutical ingredient.

The extrudate can comprise from 10% by weight to 60% by weight of the active pharmaceutical ingredient. The extrudate can comprise from 15% by weight to 55% by weight of the active pharmaceutical ingredient. The extrudate can comprise from 20% by weight to 50% by weight of the active pharmaceutical ingredient. The extrudate can comprise from 25% by weight to 45% by weight of the active pharmaceutical ingredient. The extrudate can comprise from 30% by weight to 40% by weight of the active pharmaceutical ingredient.

Typically, the combination of polymers is miscible with the active pharmaceutical ingredient.

The first polymer of the combination of at least two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 30° C. and 60° C. The polymer can have a glass transition temperature of between 40° C. and 50° C.

Typically, the first polymer of the combination of two polymers can be selected from polymethacrylates including but not limited to amino methacrylate copolymer, amino-methyl methacrylate, methacrylic acid methyl methacrylate, dimethylaminoethyl methacrylate co-polymer. A preferred polymer is dimethylaminoethyl methacrylate co-polymer.

The first polymer can be a combination of polymers selected from polymethacrylates including but not limited to amino methacrylate copolymer, amino-methyl methacrylate, methacrylic acid methyl methacrylate, dimethylaminoethyl methacrylate co-polymer.

The first polymer of the combination of two polymers can be present at a level of 10-60% by weight of the extrudate.

The first polymer of the combination of two polymers can be present at a level of 10-25% by weight of the extrudate.

Alternatively, the first polymer of the combination of two polymers can be present at a level of 35-50% by weight of the extrudate.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of less than 140° C. The polymer can have a glass transition temperature of between 80° C. and 140° C. The polymer can have a glass transition temperature of between 90° C. and 110° C.

Alternatively, the second polymer of the combination of two polymers can be selected from polymers having a glass transition temperature of at least 55° C. The polymer can have a glass transition temperature of at least 60° C. The polymer can have a glass transition temperature of at least 65° C. The polymer can have a glass transition temperature of less than 70° C. The polymer can have a glass transition temperature of less than 75° C. The polymer can have a glass transition temperature of less than 80° C. The polymer can have a glass transition temperature of between 55° C. and 80° C. The polymer can have a glass transition temperature of between 60° C. and 75° C. The polymer can have a glass transition temperature of between 62° C. and 70° C.

The solidified melt extrudate can comprise an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C. More preferably, the solidified melt extrudate comprises an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can consist essentially of an active pharmaceutical ingredient selected from expectorants, antitussives and erectile dysfunction medicaments, a combination of two polymers wherein the first polymer can have a glass transition temperature of between 30° C. and 60° C. and the second polymer can have a glass transition temperature of between 80° C. and 140° C.

The solidified melt extrudate can consist essentially of an active pharmaceutical ingredient selected from expectorants, antitussives and erectile dysfunction medicaments, a combination of two polymers wherein the first polymer can have a glass transition temperature of between 40° C. and 50° C. and the second polymer can have a glass transition temperature of between 90° C. and 110° C.

The solidified melt extrudate can comprise an analgesic other than an NSAID, a combination of two polymers wherein the first polymer can have a glass transition temperature of between 30° C. and 60° C. and the second polymer can have a glass transition temperature of between 55° C. and 80° C. More preferably, the solidified melt extrudate comprises an analgesic other than an NSAID, a combination of two polymers wherein the first polymer can have a glass transition temperature of between 40° C. and 50° C. and the second polymer can have a glass transition temperature of between 65° C. and 70° C.

The solidified melt extrudate can consist essentially of an analgesic other than an NSAID, a combination of two polymers wherein the first polymer can have a glass transition temperature of between 30° C. and 60° C. and the second polymer can have a glass transition temperature of between 55° C. and 80° C.

The solidified melt extrudate can consist essentially of an analgesic other than an NSAID, a combination of two polymers wherein the first polymer can have a glass transition temperature of between 40° C. and 50° C. and the second polymer can have a glass transition temperature of between 65° C. and 70° C.

The second polymer of the combination of two polymers can be selected from hydroxypropylmethylcelluloses, polyoxyethylene-polypropylene glycols (also known as poloxamers), polyvinylpyrrolidone, polyvinylpyrrolidone-vinyl acetate co-polymer and polyvinyl caprolactam-polyvinyl acetate-polyethylene glycol graft copolymer and polyvinyl alcohol. The second polymer can be hydroxypropylmethyl cellulose, hydroxypropylmethyl cellulose acetate succinate, poloxamer 188, poloxamer 407, polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons. Preferred polymers are polyvinylpyrrolidone K12, polyvinylpyrrolidone-vinyl acetate co-polymer (PVPVA64) or a polyvinyl alcohol having a molecular weight of 30,000-35,000 Daltons.

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone K12, polyvinylpyrrolidone-vinyl acetate co-polymer (PVPVA64), polyvinylpyrrolidone 12 PF, polyvinylpyrrolidone 17 PF or polyvinyl alcohol. The polyvinyl alcohol can have a molecular weight of 30,000-35,000 Daltons.

The second polymer is selected such that the extrudate has a tensile strength of 5-50 N/mm².

The second polymer of the combination of two polymers can be present at a level of 10-60% by weight of the extrudate.

The second polymer of the combination of two polymers can be present at a level of 10-25% by weight of the extrudate. The second polymer of the combination of two polymers can be present at a level of 20-25% by weight of the extrudate.

Alternatively, the second polymer of the combination of two polymers can be present at a level of 30-60% by weight of the extrudate. The second polymer of the combination of two polymers can be present at a level of 50-60% by weight of the extrudate.

The combination of the first and second polymers can be dimethylaminoethyl methacrylate co-polymer and polyvinylpyrrolidone-vinyl acetate co-polymer (PVPVA64). The combination of first and second polymers is dimethylaminoethyl methacrylate co-polymer and polyvinylpyrrolidone K12. The combination of the first and second polymers can be dimethylaminoethyl methacrylate co-polymer and polyvinyl alcohol.

The solidified melt extrudate can comprise 10%-60% by weight of an active pharmaceutical ingredient selected from expectorants, antitussives, mucolytics, erectile dysfunction medicaments and analgesics other than NSAIDs and a combination of 10%-60% by weight of the first polymer and 10%-60% by weight of the second polymer.

The ratio of the first polymer and the second polymer of the combination of two polymers can be selected to be from 6:1 to 1:6. The ratio of the first polymer and the second polymer of the combination of two polymers can be selected to be from 5:1 to 1:5. The ratio of the first polymer and the second polymer of the combination of two polymers can be selected to be from 1:1 to 1:3. Alternatively, the ratio of the first polymer to the second polymer can be selected to be 1:4 to 1:5.

The ratio of the of the active pharmaceutical ingredient, the first polymer and the second polymer can be from 1:0.2:0.2-1:2:2.

The ratio of the of the active pharmaceutical ingredient, the first polymer and the second polymer can be from 1:0.2:0.8-1:0.8:0.2. The ratio of the of the active pharmaceutical ingredient, the first polymer and the second polymer can be about 1:0.4:0.6.

Alternatively, the ratio of the of the active pharmaceutical ingredient, the first polymer and the second polymer can be from 1:0.5:1.5-1:0.8:1.8.

Alternatively, the ratio of the of the active pharmaceutical ingredient, the first polymer and the second polymer can be from 1:1.5:1.5-1:1.5:1.5. The ratio of the of the active pharmaceutical ingredient, the first polymer and the second polymer can be about 1:1.16:1.16.

The ratio of the of the active pharmaceutical ingredient, the first polymer and the second polymer can be from 1:0.45:0.35-1:0.65:0.55. The active pharmaceutical ingredient can be selected to be paracetamol, dextromethorphan or sildenafil. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:1.2:0.2-1:1.3:0.3. The active pharmaceutical ingredient can be selected to be paracetamol or dextromethorphan. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The ratio of the of the active pharmaceutical ingredient, the first polymer and the second polymer can be from 1:0.2:1.2-1:0.7:1.7. The active pharmaceutical ingredient can be selected to be guaifenesin or paracetamol. The first polymer can be dimethylaminoethyl methacrylate co-polymer. The second polymer can be polyvinylpyrrolidone-vinyl acetate co-polymer (PVPVA64) or polyvinyl alcohol having a molecular weight of 30,000-35,000 Daltons.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can comprise 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol and a combination of 10%-50% by weight of the first polymer and 10%-50% by weight of the second polymer. The solidified melt extrudate can comprise 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of guaifenesin and a combination of 15%-25% by weight of the first polymer and 45%-55% by weight of the second polymer. The solidified melt extrudate can comprise 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can comprise 40%-50% by weight of dextromethorphan and a combination of 20%-50% by weight of the first polymer and 10%-30% by weight of the second polymer. The solidified melt extrudate can comprise 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can comprise 30%-50% by weight of sildenafil and a combination of 20%-40% by weight of the first polymer and 30%-40% by weight of the second polymer. The solidified melt extrudate can comprise 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of the first polymer and 10%-50% by weight of the second polymer.

The solidified melt extrudate can consist essentially of 30%-50% by weight of an active pharmaceutical ingredient selected from expectorants, antitussives, mucolytics, erectile dysfunction medicaments and analgesics other than NSAIDs and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can consist essentially of 40%-50% by weight of paracetamol and a combination of 10%-50% by weight of the first polymer and 10%-50% by weight of the second polymer. The solidified melt extrudate can consist essentially 40%-50% by weight of paracetamol and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of guaifenesin and a combination of 15%-25% by weight of the first polymer and 45%-55% by weight of the second polymer. The solidified melt extrudate can consist essentially 30%-50% by weight of guaifenesin and a combination of 15%-25% by weight of dimethylaminoethyl methacrylate co-polymer and 45%-55% by weight of polyvinylpyrrolidone-vinyl acetate co-polymer (PVPVA64).

The solidified melt extrudate can consist essentially of 40%-50% by weight of dextromethorphan and a combination of 20%-50% by weight of the first polymer and 10%-30% by weight of the second polymer. The solidified melt extrudate can consist essentially 40%-50% by weight of dextromethorphan and a combination of 10%-50% by weight of dimethylaminoethyl methacrylate co-polymer and 10%-50% by weight of polyvinylpyrrolidone-vinyl acetate co-polymer (PVPVA64).

The solidified melt extrudate can consist essentially of 30%-50% by weight of sildenafil and a combination of 20%-40% by weight of the first polymer and 30%-40% by weight of the second polymer. The solidified melt extrudate can consist essentially 30%-50% by weight of sildenafil and a combination of 20%-40% by weight of dimethylaminoethyl methacrylate co-polymer and 30%-40% by weight of polyvinylpyrrolidone-vinyl acetate co-polymer (PVPVA64).

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of the first polymer and 10%-50% by weight of the second polymer.

The solidified melt extrudate can comprise 30%-50% by weight of an expectorant, an antitussive, a mucolytic, an erectile dysfunction medicament or an analgesic other than an NSAID as the sole active pharmaceutical ingredient and a combination of 10%-50% by weight of dimethylaminoethyl methacrylate co-polymer and 10%-50% by weight of polyvinylpyrrolidone-vinyl acetate co-polymer (PVPVA64) or polyvinyl alcohol having a molecular weight of 30,000-35,000 Daltons.

The solidified melt extrudate can comprise 40%-50% by weight of paracetamol as the sole active pharmaceutical ingredient and a combination of 10%-50% by weight of the first polymer and 10%-50% by weight of the second polymer. The solidified melt extrudate can comprise 40%-50% by weight of paracetamol as the sole active pharmaceutical ingredient.

The composition can include one or more excipients such as glidants, pH modifiers, channelling agents, disintegrants, and surfactants.

The glidants can be selected from silicon dioxide (syloid G, syloid 244 FP), talc, magnesium oxide, glycerol monostearate, sodium stearyl fumarate, magnesium stearate.

The glidants can be included at a level of 1-5 w/w %.

The pH modifiers can be selected from malic acid, citric acid, tartaric acid and acetic acid.

The pH modifiers can be included at a level of 1-10 w/w %.

The channelling agents can be selected from mannitol, xylitol, calcium phosphate and calcium carbonate.

The channelling agents can be included at a level of 5-10 w/w %.

The disintegration aids can be selected from crospovidone, microcrystalline cellulose, sodium starch glycolate and croscarmellose sodium.

The surfactants can be selected from polysorbates, sodium lauryl sulphate, poloxamers or lecithin. A preferred surfactant is polysorbate 80 (Tween 80). Polysorbate 80 can be present as a level of 1-5 w/w %.

One or more processing aids such as talc, magnesium silicate and glyceryl monostearate can be added prior to melt extrusion or post-melt extrusion.

One or more additional processing aids can be added post-melt extrusion selected from microcrystalline cellulose, crospovidone, carrageenan, chitosan, pectinic acid, glycerides, beta-cyclodextrin and cellulose derivatives, sorbitol and mannitol and isomers thereof.

The combination of polymers is soluble at a pH of about 1 to about 5. Preferably, the combination of polymers is soluble at a pH of about 2 to about 4.

Preferably the difference in Hansen solubility parameter between the active pharmaceutical ingredient and one or both of the first polymer and the second polymer is less than 10 MPa^{1/2}. More preferably the difference in Hansen solubility parameter between the active pharmaceutical ingredient and one or both of the first polymer and the second polymer is less than 7 MPa^{1/2}.

The extrudate is typically prepared using a hot melt extrusion process.

When the active pharmaceutical ingredient is selected from expectorants the melt extrusion process can be conducted at a temperature of between 70° C. and 85° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 70° C. to 85° C. and at an extruder screw speed from 90 rpm to 110 rpm.

When the active pharmaceutical ingredient is selected from antitussives the melt extrusion process can be conducted at a temperature of between 100° C. and 120° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 100° C. to 120° C. and at an extruder screw speed from 90 rpm to 110 rpm.

When the active pharmaceutical ingredient is selected from erectile dysfunction medicaments, the melt extrusion process can be conducted at a temperature of between 110° C. and 140° C. The screw speed of the extruder can be selected from 190 revolutions per minute (rpm) to 210 revolutions per minute (rpm). The screw speed of the extruder can be selected to be about 200 rpm. The melt extrusion process can be carried out at a preferred temperature from 110° C. to 140° C. and at an extruder screw speed from 190 rpm to 210 rpm.

When the active pharmaceutical ingredient is selected from analgesics other than NSAIDs, the melt extrusion process can be conducted at a temperature of between 110° C. and 150° C. The screw speed of the extruder can be selected from 90 revolutions per minute (rpm) to 110 revolutions per minute (rpm). The screw speed of the extruder can be selected to be about 100 rpm. The melt extrusion process can be carried out at a preferred temperature from 110° C. to 150° C. and at an extruder screw speed from 90 rpm to 110 rpm.

The extrusion processing conditions can be selected such that the torque is less than or equal to 15 Nm. The extrusion conditions can be selected such that the torque is more than or equal to 3 Nm.

Preferably the extrusion conditions are selected such that the torque is less than or equal to 15 Nm and equal to or more than 3 Nm.

The solidified melt extrudate comprises an active pharmaceutical ingredient selected from expectorants and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from expectorants and wherein the extrudate can be prepared using a hot melt extrusion process at a temperature between 70° C. and 85° C. and wherein the extruder screw speed can be about 100 rpm.

The solidified melt extrudate comprises an active pharmaceutical ingredient selected from antitussives and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from antitussives and wherein the extrudate can be prepared using a hot melt extrusion process at a temperature between 100° C. and 120° C. and wherein the extruder screw speed can be about 100 rpm.

The solidified melt extrudate comprises an active pharmaceutical ingredient selected from erectile dysfunction medicaments and a combination of dimethylaminoethyl methacrylate co-polymer and polyvinylpyrrolidone-vinyl acetate co-polymer (PVPVA64) wherein the extrudate can comprise from 30%-50% by weight of the active pharmaceutical ingredient selected from erectile dysfunction medicaments and wherein the extrudate can be prepared using a hot melt extrusion process at a temperature from 110° C. to 140° C. and at an extruder screw speed from 190 rpm to 210 rpm.

The solidified melt extrudate comprises an analgesic other than an NSAID and a combination of dimethylaminoethyl methacrylate co-polymer and polyvinyl alcohol wherein the extrudate can comprise from 30%-50% by weight of the analgesic other than an NSAID wherein the extrudate can be prepared using a hot melt extrusion process at a temperature between 110° C. and 150° C. and wherein the extruder screw speed can be about 100 rpm.

The extrudate can be comminuted into granules that are suitable for incorporation into an oral dosage form such as oral liquid suspensions, tablets, orodispersible tablets, direct to mouth granules, capsules, lyophilates.

The solidified melt extrudates of the present invention are prepared using a single melt extrusion process, i.e. the active pharmaceutical ingredient and the combination of the two polymers are simultaneously fed and extruded in a single hot melt extrusion process.

For the avoidance of doubt the term “torque” refers to the force needed to cause the screw in the extruder to turn.

The Hansen solubility parameter is a numerical value used to indicate the relative solvency of a particular material. It is typically used to determine if a particular material will dissolve in another material, and is well-known to the person skilled in the art.

In the context of the present invention the terms “granule” and “granules” as used herein refer to discrete particle or particles and includes pellets, powders or spheres.

Embodiments of the present invention will now be described, by way of example only, with reference to the accompanying Figures in which:

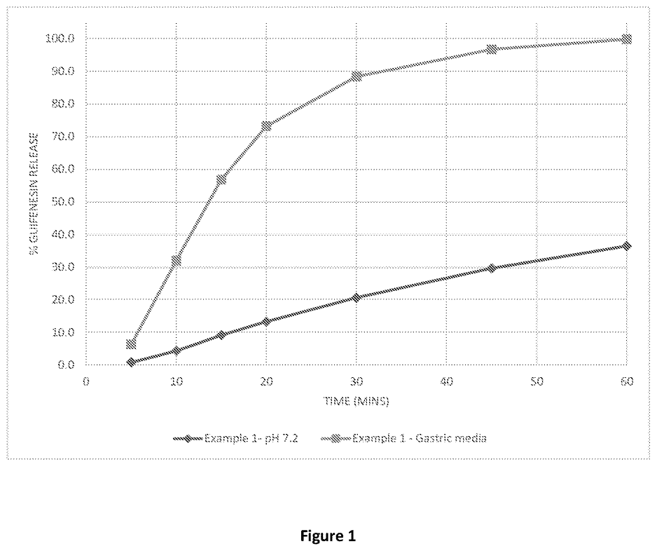

FIG. 1 illustrates the dissolution profile for a guaifenesin-containing composition (Example 1) of the present invention at a pH of 1.2 and a pH of 7.2;

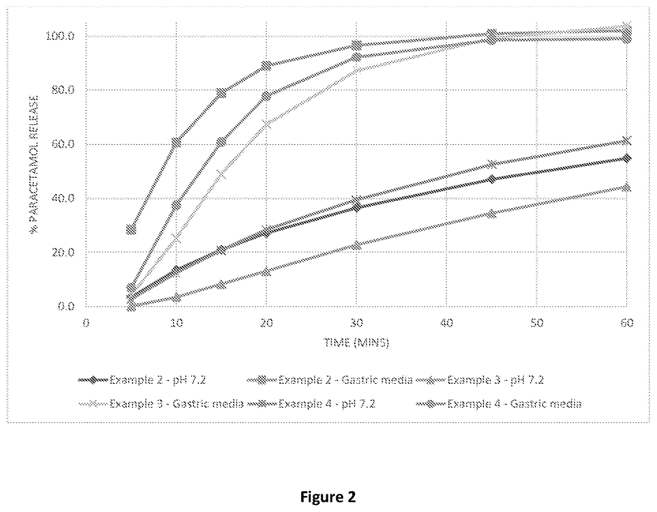

FIG. 2 illustrates the dissolution profile for a paracetamol-containing composition (Examples 2-4) of the present invention at a pH of 1.2 and a pH of 7.2; and

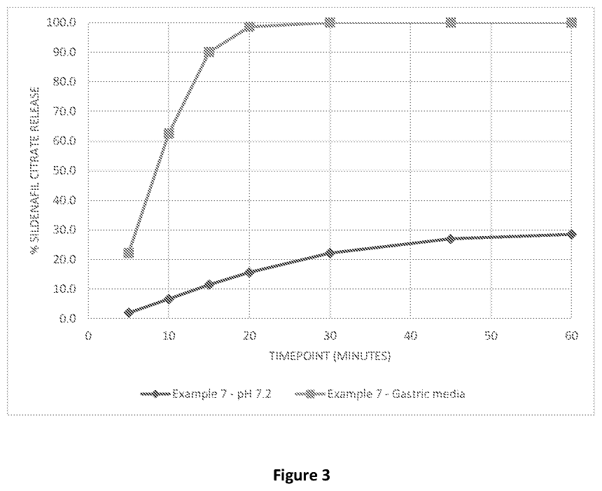

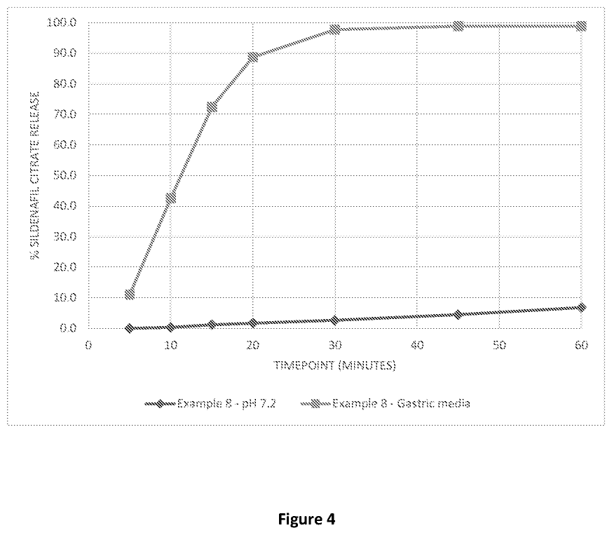

FIGS. 3 & 4 illustrate the dissolution profile for a sildenafil-containing composition (Examples 7 & 8) of the present invention at a pH of 1.2 and a pH of 7.2.

The compositions of the present invention can be made in the following way.

The active pharmaceutical ingredient, Eudragit (EPO) and either polyvinyl alcohol or PVPVA64 were extruded using a Leistritz Nano 16 (Somerville, NJ, USA), which is a co-rotating twin screw extruder (screw diameter 16 mm) with three heating zones and a die zone. Barrel temperature was kept at 85° C. (for GGE, DM & SILD) and 110-150° C. for paracetamol. The screw speed was 100 rpm. Full conveying screw geometry was used in all extrusion experiments. Extrudates were stored in glass vials and used for further characterisation.

Each formulation ingredient was sieved and then blended. The formulation pre-mix was transferred to a feeder. The pre-mix was fed into the extruder at a rate of 7 g/min. The feeding zone has a water jacket cooling system kept at 5° C. The formulation was heated in the extruder barrel and the molten extrudate was passed on to a 2 m conveyor belt cooled with compressed air prior to milling. The samples can be milled at room temperature if required.

Example formulations are shown below in Table 1:

| | Ex 1 | Ex 2 | Ex 3 | Ex4 | Ex 5 | Ex 6 | Ex 7 | Ex 8 |
|---|---|---|---|---|---|---|---|---|
| Material | (% w/w) | (% w/w) | (% w/w) | (% w/w) | (% w/w) | (% w/w) | (% w/w) | (% w/w) |
| Paracetamol | — | 40 | 40 | 50 | — | — | — | — |
| Guaifenesin | 30 | — | — | — | — | — | — | — |
| Dextromethorphan | — | — | — | — | 40 | 50 | — | — |
| Sildenafil | — | — | — | — | — | — | 50 | 30 |
| Eudragit | 20 | 10 | 50 | 30 | 50 | 25 | 25 | 35 |
| Kollidon VA64 | 50 | — | — | — | 10 | 25 | 25 | 35 |
| Polyvinyl Alcohol | — | 50 | 10 | 20 | — | — | — | — |
| TOTAL | 100 | 100 | 100 | | 100 | 100 | 100 | 100 |

The dissolution studies were performed as follows. USP Type II apparatus as defined by the United States Pharmacopoeia (USP) was used. The procedure followed was that set out in USP 711.

Two dissolution baths were used-one bath was provided with a 900 ml of a phosphate buffer at a pH of 7.2 and a temperature of 37° C., and the other bath was provided with 900 ml of simulated gastric fluid (pH 1.2) and a temperature of 37° C. The paddles in each bath were rotated at 50 RPM throughout.

Simulated gastric fluid was prepared as per EP 9.0 5.17.1. which involved dissolving 2 g sodium chloride in 80 ml hydrochloric acid and 920 ml deionised water.

Each dissolution bath had an automated in-line UV sampler which allowed samples to be taken at set time points. The samples were then run through a UV/Vis spectrometer to calculate the percentage of guaifenesin or paracetamol dissolved. Samples were taken at 5, 10, 15, 20, 30, 45 and 60 minutes and the UV was set to take readings at 275 nm for paracetamol, 280 nm for guaifenesin and 290 nm for sildenafil citrate.

As can be seen, in each of the Examples there was very little release of the active pharmaceutical ingredient at pH 7.2 (oral pH) and immediate release at a pH of about 1.2/gastric media.

An advantage of the present invention is that there is provided a composition with excellent taste-masking properties as a result of the solubility of active pharmaceutical ingredient being minimised at oral pH. The composition can be incorporated into an oral dosage form without the need for the inclusion of an additional agent to mask the unpleasant or bitter taste of the active pharmaceutical ingredient. The formulation of the present invention also has an improved dissolution profile.

The present invention is not intended to be limited to the exemplary embodiments described herein. Further modifications can be made without departing from the scope of the invention described herein.

1-57. (canceled)

58. A taste-masked composition in the form of a solidified melt extrudate comprising an active pharmaceutical ingredient and pharmaceutically acceptable salts or esters thereof and a combination of a first polymer and a second polymer wherein the composition has a solubility of less than 5% after 5 mins in an aqueous medium at a pH of between 6 and 7.5 and a solubility of more than 5% after 5 mins in an aqueous medium at a pH of between 1 and 5 and wherein the active pharmaceutical agent is selected from expectorants, antitussives, mucolytics, erectile dysfunction medicaments, or analgesics other than NSAIDs.

59. The composition as claimed in claim 58, wherein the active pharmaceutical ingredient is selected to be guaifenesin, paracetamol, dextromethorphan or sildenafil.

60. The composition as claimed in claim 58, wherein the extrudate comprises from 10% by weight to 60% by weight of the active pharmaceutical ingredient.

61. The composition as claimed in claim 58, wherein the first polymer of the combination has a glass transition temperature of between 30° C. and 60° C.

62. The composition as claimed in claim 58, wherein the second polymer of the combination has a glass transition temperature of between 80° C. and 140° C.

63. The composition as claimed in claim 58, wherein the solidified melt extrudate comprises the active pharmaceutical ingredient selected from expectorants, antitussives or erectile dysfunction medicaments, and the combination of first and second polymers wherein the first polymer has a glass transition temperature of between 30° C. and 60° C. and the second polymer has a glass transition temperature of between 80° C. and 140° C.

64. The composition as claimed in claim 58, wherein the first polymer of the combination is selected from amino methacrylate copolymer, amino-methyl methacrylate, methacrylic acid methyl methacrylate, or dimethylaminoethyl methacrylate co-polymer.

65. The composition as claimed in claim 58, wherein the first polymer of the combination is present at a level 10-60% by weight of the extrudate.

66. The composition as claimed in claim 58, wherein the second polymer of the combination is selected from polyvinylpyrrolidone, polyvinylpyrrolidone-vinyl acetate co-polymer and polyvinyl caprolactam-polyvinyl acetate-polyethylene glycol graft copolymer and polyvinyl alcohol.

67. The composition as claimed in claim 58, wherein the second polymer of the combination is present at a level of 10-60% by weight of the extrudate.

68. The composition as claimed in claim 58, wherein the second polymer of the combination is present at a level of 50-60% by weight of the extrudate.

69. The composition as claimed in claim 58, wherein the combination of the first and second polymers is dimethylaminoethyl methacrylate co-polymer and polyvinylpyrrolidone-vinyl acetate co-polymer (PVPVA64).

70. The composition as claimed in claim 58, wherein the combination of first and second polymers is dimethylaminoethyl methacrylate co-polymer and polyvinylpyrrolidone K12.

71. The composition as claimed claim 58, wherein the combination of the first and second polymers is dimethylaminoethyl methacrylate co-polymer and polyvinyl alcohol.

72. The composition as claimed in claim 58, wherein the ratio of the active pharmaceutical ingredient, the first polymer and the second polymer is from 1:0.2:0.2-1:2:2.

73. The composition as claimed in claim 58, wherein the second polymer is selected to be polyvinylpyrrolidone-vinyl acetate co-polymer (PVPVA64) or polyvinyl alcohol having a molecular weight of 30,000-35,000 Daltons.

74. The use or composition as claimed in claim 58, wherein the ratio of the active pharmaceutical ingredient, the first polymer and the second polymer is from 1:1.2:0.2-1:1.3:0.3 and the active pharmaceutical ingredient is selected to be paracetamol or dextromethorphan.

75. The composition as claimed in claim 58, wherein the ratio of the active pharmaceutical ingredient, the first polymer and the second polymer is from 1:1.1:1.1-1:1.25:1.25 and the active pharmaceutical ingredient is sildenafil.

76. The composition as claimed in claim 58, wherein the solidified melt extrudate comprises 30%-50% by weight of the active pharmaceutical ingredient selected from expectorants, antitussives, mucolytics, erectile dysfunction medicaments, or analgesics other than NSAIDs and a combination of 10%-50% by weight of the first polymer and 10%-50% by weight of the second polymer.

77. The composition as claimed in claim 76, wherein the solidified melt extrudate comprises 30%-50% by weight of the active pharmaceutical ingredient selected from expectorants, antitussives, mucolytics, erectile dysfunction medicaments, or analgesics other than NSAIDs and a combination of 10%-50% by weight of dimethylaminoethyl methacrylate co-polymer as the first polymer and 10%-50% by weight of polyvinylpyrrolidone-vinyl acetate co-polymer (PVPVA64) or polyvinyl alcohol having a molecular weight of 30,000-35,000 Daltons as the second polymer.
